(19) United States
(12) Patent Application Publication
(10) Pub. No.: US 2023/0172864 A1
(43) Pub. Date: Jun. 8, 2023

(54) SOFTGEL CAPSULES

(71) Applicant: R.P. Scherer Technologies, LLC, Carson City, NV (US)

(72) Inventor: Rickey Steve Shelley, Largo, FL (US)

(21) Appl. No.: 17/910,999

(22) PCT Filed: Mar. 18, 2021
(86) PCT No.: PCT/US21/22918

§ 371 Date: Sep. 12, 2022

Related U.S. Application Data
(60) Provisional application No. 62/991,354, filed on Mar. 18, 2020.

(51) Int. Cl. A61K 9/48; A61K 31/198

(52) U.S. Cl. CPC A61K 9/4825; A61K 9/4858; A61K 9/4816; A61K 31/198

(57) ABSTRACT

Disclosed in certain embodiments is a softgel capsule comprising (a) a fill material; and (b) a shell composition, wherein the fill material comprises at least 10% of a liquid medium, and wherein the shell composition comprises gelatin and a pH dependent polymer that solubilizes at a pH of less than about 6.0.

### RELATED APPLICATIONS

This application is a National Stage entry of International Application No. PCT/US2021/022918, filed Mar. 18, 2021, which claims the benefit of U.S. Provisional Patent Application No. 62/991,354 filed Mar. 18, 2020, which is incorporated by reference herein.

### FIELD OF THE INVENTION

The present invention relates to softgel capsules, wherein the gelatin-based shell compositions can encapsulate a fill composition with a high liquid content (e.g., water).

### BACKGROUND OF THE INVENTION

Soft capsules, in particular, soft gelatin capsules (or softgel capsules), provide a dosage form which is more readily accepted by patients, since the capsules are easy to swallow and need not be flavored in order to mask any unpleasant taste of the active agent. Softgel encapsulation of drugs further provides the potential to improve the bioavailability of the pharmaceutical agents. For example, active ingredients may be rapidly released in liquid form as soon as the gelatin shell ruptures.

Softgels have limitations as to the amount and selection of liquid mediums in the fill material that suspend or solubilize active agents. Many of these mediums solubilize the softgel upon storage. Other acceptable mediums can solubilize the Softgel if they exceed a threshold amount. This limits the ability to provide an acceptable Softgel across varying active pharmaceutical ingredients.

Also, in the cosmetic industry, there are certain fill formulations that require higher percentages of water for lotions and creams for topical application. By being able to encapsulate higher concentrations of water into the fill, this would open up a greater range of applications for the softgel in the cosmetic industry.

Accordingly, there is currently a need for an improved softgel that can be used to formulate varied fill materials.

### OBJECTS AND SUMMARY OF THE INVENTION

It is an object of certain embodiments of the present invention to provide a softgel capsule that maintains its integrity with a high liquid content.

It is an object of other embodiments of the present invention to provide methods of treating a disease or condition (e.g., thyroid insufficiency) with a softgel capsule as disclosed herein.

It is an object of other embodiments of the present invention to provide methods of packaging and delivering cosmetic formulations with softgels as disclosed herein.

It is an object of further embodiments of the present invention to provide methods of preparing the softgel capsule as disclosed herein

One or more of the above objects and others, may be met by the present invention which in certain embodiments is directed to a softgel capsule comprising (a) a fill material; and (b) a shell composition, wherein the fill material comprises at least 10% of a liquid medium selected from water, a polyol, a glycol, an alcohol or a combination thereof, and wherein the shell composition comprises gelatin and a pH dependent polymer that solubilizes at a pH of less than about 6.0.

In other embodiments, the present invention is directed to a method of treating a disease or condition (e.g., by providing thyroid replacement therapy) comprising administering a softgel capsule as disclosed herein.

In further embodiments, the present invention is directed to a process of preparing a softgel capsule as disclosed herein comprising the steps of preparing the fill material with at least 10% of a liquid medium and encapsulating the fill material with a shell composition comprising gelatin and a pH dependent polymer that solubilizes at a pH of less than about 6.0.

### DETAILED DESCRIPTION OF THE INVENTION

The present invention advances the state of the art by developing softgel capsules capable of encapsulating a wider variety of fill compositions. Softgels have limitations as to the amount of different hydrophilic solvents that can be encapsulated into the gelatin shell. Solvents such as water, glycerin, and propylene glycol can be used to solubilize certain hydrophilic APIs. Because of these limitations, it is difficult to develop softgel fill formulations for these compounds. By developing a gelatin shell containing gelatin and a pH dependent polymer as disclosed herein, there is provided an improved softgel that can increase the range of fill compositions, APIs and excipients to be encapsulated.

Also, in the cosmetic industry, there are certain fill formulations that require higher percentages of water for lotions and creams for topical application. By virtue of the present invention, softgels can be used to encapsulate higher concentrations of water or other liquid mediums such as glycerin, polyethylene glycol and ethanol into the fill, and would open up a greater range of applications for softgels in the cosmetic industry.

Furthermore, typical softgel capsules are unable to contain fill compositions and/or active agents having extreme acidic and basic pH. For example, a fill formulation with a pH of 2.5 can hydrolyse gelatin (leading to leakage). Also, fill materials with a pH of greater than 9 can have a tanning effect on the gelatin. A tanning process involves crosslinking of gelatin, which results in hardening of the shell. The shell becomes insoluble in water and resistant to digestion by gastrointestinal enzymes: trypsin and chymotrypsin. In some embodiments, it is desirable to encapsulate a fill formulation having a pH of greater than about 4, but that does not dissolve or interact with the softgel (e.g., to prevent leakage and/or release of the fill formulation) and so that the softgel may appropriately dissolve in the digestive tract (e.g., before it reaches the large intestine). By virtue of the present invention, softgels can be used to encapsulate fill formulations and/or active agents having a pH of greater than about 4, greater than about 5, greater than about 6, greater than about 7, greater than about 8, greater than about 9, or about 4 to about 14, about 4 to about 6, about 5 to about 7, about 6 to about 8, about 7 to about 9, about 8 to about 10, about 9 to about 11, about 10 to about 12, about 11 to about 13, about 12 to about 14, about 10 to about 14, about 10 to about 13, about 10 to about 12, or any individual pH or sub-range within these ranges.

As used herein, the term “pH dependent” is used to refer to the dissolution or disintegration resistant property of a substance such that dissolution or disintegration does not occur or does not substantially occur in a basic environment, e.g., for a time period of at least about 1 hour. For example, the embodiments described herein include a pH dependent shell composition that preferentially dissolves in biological, artificial or simulated gastric fluid as compared to biological, artificial or simulated intestinal fluid. As used herein, “pharmaceutically active ingredient” or “active pharmaceutical ingredient (API)” refers to a drug or compound that may be used in the diagnosis, cure, mitigation, treatment, or prevention of a condition. The term “condition” or “conditions” refers to those medical conditions that can be treated or prevented by administration to a subject of an effective amount of an active agent. Exemplary non-limiting conditions that may benefit from softgel capsules may include, without limitations, capsules containing thyroid replacement such as levothyroxine or a pharmaceutically acceptable salt thereof.

As used herein, the term “active ingredient” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

Any pharmaceutically active ingredient may be used for purposes of the present invention, including both those that are water-soluble and those that are poorly soluble in water. Suitable pharmaceutically active ingredients include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In certain embodiments, the active ingredient is a thyroid replacement, e.g., levothyroxine (T4), liothyronine (T3) or pharmaceutically acceptable salts thereof such as levothyroxine sodium and liothyronine sodium. In a particular embodiments, the softgel includes levothyroxine sodium in an amount of from 0.01 mg to about 0.5 mg. In particular embodiments, the softgel includes levothyroxine sodium in an amount of 0.013 mg, 0.025 mg, 0.05 mg, 0.075 mg, 0.088 mg, 0.1 mg, 0.112 mg, 0.125 mg, 0.137 mg, 0.15 mg, 0.175 mg, or 0.2 mg.

In some embodiments, the lipids in the dosage form may be selected, without limitations, from the group consisting of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, animal or vegetable fats, e.g., in their hydrogenated form, free fatty acids and mono-, di-, and tri-glycerides with C8-, C10-, C12-, C14-, C16-, C18-, C20- and C22-fatty acids, and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-camitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Camitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lomoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2,4,6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

In some embodiments, the active agent (or the fill material containing the active agent dissolved therein) has a pH of greater than about 4. In one or more embodiments, the pH of the active agent (or the fill material containing the active agent dissolved therein) is greater than about 4, greater than about 5, greater than about 6, greater than about 7, greater than about 8, greater than about 9, or about 4 to about 14, about 4 to about 6, about 5 to about 7, about 6 to about 8, about 7 to about 9, about 8 to about 10, about 9 to about 11, about 10 to about 12, about 11 to about 13, about 12 to about 14, about 10 to about 14, about 10 to about 13, about 10 to about 12, or any individual pH or sub-range within these ranges.

As used herein, the terms “therapeutically effective” and an “effective amount” refer to the amount of active agent or the rate at which it is administered which is needed to produce a desired therapeutic result.

As used herein, “shell” or “shell composition” refers to the shell of a softgel capsule which encapsulates a fill material.

As used herein, “free or substantially free,” refers to a composition that comprises less than about 1 wt %, less than about 0.5 wt %, less than about 0.25 wt %, less than about 0.1 wt % , less than about 0.05 wt %, less than about 0.01 wt %, or 0 wt % of said component.

All references to wt % throughout the specifications and the claims refer to the weight of the component in reference to the weight of the entire composition and may also be designated as w/w.

As used herein, “fill material” or “fill” refers to the composition that is encapsulated by the pH dependent capsule shell and optionally contains at least one pharmaceutically active ingredient.

As used herein, “about” refers to any values that are within a variation of ±10%, such that “about 10” would include from 9 to 11. As used herein, “a,” “an,” or “the” refers to one or more, unless otherwise specified. Thus, for example, reference to “an excipient” includes a single excipient as well as a mixture of two or more different excipients, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods

In certain embodiments, the invention is directed to a softgel capsule comprising (a) a fill material; and (b) a shell composition, wherein the fill material comprises at least 10% of a liquid medium selected from water, a polyol, a glycol or a combination thereof, and wherein the shell composition comprises gelatin and a pH dependent polymer that solubilizes at a pH of less than about 6.0

The softgel capsule may optionally comprise an active agent dissolved or suspended in the liquid medium

In certain embodiments, the pH dependent polymer solubilizes at a pH of less than about 5.5, less than about 5.0, less than about 4.5 or less than about 4.0.

In certain embodiments, the pH dependent polymer is an acrylic polymer or a cellulosic polymer.

In certain embodiments, the pH dependent polymer is an acrylic polymer such as an amino methacrylate copolymer.

In certain embodiments, the shell composition comprises the pH dependent polymer in an amount (w/w) from about 1% to about 60%, from about 5% to about 50%, from about 10% to about 40% or about 15% to about 35%.

In certain embodiments, the shell composition comprises the gelatin in an amount (w/w) from about 5% to about 75%, from about 10% to about 60%, from about 15% to about 50% or about 20% to about 40%.

In certain embodiments, the shell composition further comprises a solubilizing agent such as an organic acid, e.g., oxalic acid, malonic acid, succinic acid, glutaric acid, adipic acid, fumaric acid, maleic acid, phthalic acid, isophthalic acid, terephthalic acid, formic acid, acetic acid, propionic acid, butyric acid, valeric acid, cinnamic acid, lactic acid, benzoic acid, salicylic acid, gallic acid or toluic acid. In a particular embodiment, the organic acid is lactic acid.

In certain embodiments, the shell composition comprises the solubilizing agent in an amount (w/w) from about 0.0001% to about 1%, from about 0.001% to about 0.5%, or about 0.005% or about 0.1%.

In certain embodiments, the shell composition further comprises a plasticizer, e.g., in an amount (w/w) of about 0.1 wt % to about 50 wt %,about 5% to about 45%, about 10% to about 40% or about 15% to about 35%.

In certain embodiments, the plasticizer is selected from glycerol, glycerin, sorbitol or combinations thereof.

In certain embodiments, the gelatin is selected from Type A gelatin, Type B gelatin or mixtures thereof.

In certain embodiments, the gelatin is selected from the group consisting of fish gelatin, hide gelatin, bone gelatin or mixtures thereof.

In certain embodiments, the liquid medium is water, a polyol, a glycol, an alcohol or a combination thereof. The polyol may be glycerol; the glycol may be polyethylene glycol and the alcohol may be ethanol.

In certain embodiments, the fill composition comprises the liquid medium in an amount of at least 20%, at least 30%, at least 40%, at least 50%, at least 60%, at least 70%, at least 80%, at least 90%, at least 95% or at least 99%. In certain embodiments, the fill composition comprises the liquid medium in an amount of from 5% to 50%, about 10% to about 40% or about 15% to about 30%.

In certain embodiments, the capsule disintegrates in less than about 60 minutes, less than about 45 minutes, less than about 30 minutes, less than about 20 minutes, less than about 10 minutes, or less than about 5 minutes in a gastric environment based on a disintegration test performed in a USP Apparatus II with paddles at a speed of 50 rpm in pH 1.2 buffer.

In certain embodiments, the capsule disintegrates in at least about one hour, at least about two hours, at least about three hours, at least about four hours, or at least about five hours in a basic medium based on a disintegration test performed in a basket-rack assembly NT-40H model apparatus in a 1000 mL beaker at about 37° C.±2° C.

Suitable fill materials optionally comprise at least one pharmaceutically active ingredient and can be made according to known methods. In addition to the at least one pharmaceutically active ingredient, suitable fill materials may comprise additional fill components such as flavoring agents, sweetening agents, coloring agents and fillers or other pharmaceutically acceptable excipients or additives such as synthetic dyes and mineral oxides. Suitable amounts of pharmaceutically active ingredient and pharmaceutically acceptable excipients can be readily determined by one of ordinary skill in the art.

In some embodiments, the fill material (with or without the active agent) within a dosage form according to the disclosure has a pH of greater than about 4. For example, the pH of the fill material is greater than about 4, greater than about 5, greater than about 6, greater than about 7, greater than about 8, greater than about 9, or about 4 to about 14, about 4 to about 6, about 5 to about 7, about 6 to about 8, about 7 to about 9, about 8 to about 10, about 9 to about 11, about 10 to about 12, about 11 to about 13, about 12 to about 14, about 10 to about 14, about 10 to about 13, about 10 to about 12, or any individual pH or sub-range within these ranges.

In an embodiment, the gelatin in the pH dependent shell composition may include Type A gelatin, Type B gelatin, a hide or skin gelatin and/or a bone gelatin used alone or in combination. In one embodiment, the gelatin is a 250 Bloom gelatin. In another embodiment, there is only one type of gelatin. In yet another embodiment, the gelatin is a combination of at least two types of gelatins. In an embodiment, the amount of gelatin in the pH dependent shell composition is about 5% to about 90%, about 10% to about 80%, about 20% to about 80%, about 40 wt % to about 80 wt %, or from about 45 wt % to about 75 wt %, or from about 50 wt % to about 70 wt %.

In one embodiment, the pH dependent capsule shell composition may comprises dextrose. In an embodiment, the amount of dextrose in the pH dependent capsule shell composition is about 0.005 wt % or about 0.01 wt % to about 4 wt %, or from about 0.1 wt % or about 0.15 wt % to about 3 wt %, or from about 0.15 wt % or about 0.2 wt % to about 2 wt %, or from about 0.1 wt % to about 0.2 wt %.

In an embodiment, the plasticizer in the pH dependent shell composition may include glycerol, glycerin, sorbitol and combinations thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In an embodiment, the amount of plasticizer in the pH dependent shell composition is about 0.1% to about 50%, about 15 wt % to about 40 wt %, or from about 20 wt % to about 35 wt %, or from about 25 wt % to about 30 wt %.

In an embodiment, the pH dependent shell composition may optionally comprise additional agents such as coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quatemary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In some embodiments, the pH dependent shell composition and/or the pH dependent softgel capsule may be tested in a disintegration test performed in a USP Apparatus II with paddles at a speed of 50 rpm in pH 1.2 buffer. The pH dependent softgel capsule according to this embodiment wherein the capsule disintegrates in less than about 60 minutes, less than about 45 minutes, less than about 30 minutes, less than about 20 minutes, less than about 10 minutes, or less than about 5 minutes.

Encapsulation of the fill material can be accomplished in any conventional manner. As an example, a rotary die encapsulation may be used.

According to an embodiment, a pH dependent softgel capsule is prepared by the process comprising the steps of: (a) preparing the fill material, said fill material optionally comprising at least one pharmaceutically active ingredient; and (b) encapsulating the fill material of step (a) in a pH dependent shell composition. The encapsulation process according to step (b) may further comprise a sub-step of preparing the pH dependent shell composition by, for example, admixing a gelatin, and pH dependent polymer and optionally a plasticizer.

### EXAMPLES

Specific embodiments of the invention will now be demonstrated by reference to the following examples. It should be understood that these examples are disclosed solely by way of illustrating the invention and should not be taken in any way to limit the scope of the present invention

Water, glycerin, and lactic acid are added to a jacketed vessel with a sweep with vacuum, pressure, and heating capabilities.

While mixing with the sweep, the Eudragit E PO (amino methacrylate copolymer) is slowly added to the jacketed vessel. The mixture is mixed with the sweep until the Eudragit E PO has completely dissolved.

While continuing to mix with the sweep, the gelatin is slowly added and mixed until a fluff forms. Turn off sweep. Deaerate the fluff

Once the fluff forms, the heating unit for the vessel is set at 70° C. After the fluff begins to melt, the sweep is turned on at approximately 15 RPM and mixed under vacuum. After all the gel has melted, approximately 2 kg of water is added to the mix. The water is mixed into the gel mass while deaerating.

After deaeration is complete, the melt is forced out of the jacketed vessel using nitrogen pressure into a jacketed gel receiver.

Two gel melts have been prepared using this process. The first gel melt was used to encapsulate three types of fill solutions:

A fill solution containing 50% water and 50% PEG 400. The fill material was easily encapsulated with good seals. The fill did not dissolve the gelatin shell. For a regular gel, a fill solution containing 50% water would be difficult to encapsulate. Poor seals would be formed if formed and the gelatin shell would begin dissolving either at the conveyor belt or in the dryer baskets.

The second fill solution contained 100% glycerin. Good seals were obtained at the encapsulation machine.

The third fill solution encapsulated was Transcutol. These softgels had good seals and maintained their shapes and appearance after removal from the shallow trays.

For the second gel melt, the following fill solution was encapsulated:

A fill solution containing 99% glycerin and 1% gelatin was encapsulated. Good seals were obtained during encapsulation and the softgels had a good appearance.

The two prepared gel melts had the following formulations:

### Gel Melt #1

### Purified Water—31.5%

### Glycerin—21.6%

### Lactic Acid—0.00736%

### Eudragit E PO—14.1%

### Gelatin—32.8%

### Gel Melt#2

### Purified Water—33.4%

### Glycerin—17.6%

### Lactic Acid—0.01%

### Eudragit E PO—14.7%

### Gelatin—34.3%

1. A softgel capsule comprising:
(a) a fill material; and
(b) a shell composition,
wherein the fill material comprises at least 10% of a liquid medium selected from water, a polyol, a glycol, an alcohol or a combination thereof, and
wherein the shell composition comprises gelatin and a pH dependent polymer that solubilizes at a pH of less than about 6.0.

2. The softgel capsule of claim 1, wherein the fill material further comprises an active agent dissolved or suspended in the liquid medium, the fill material comprising a pH of greater than about 4.

3. The softgel capsule of claim 1, wherein the pH dependent polymer solubilizes at a pH of less than about 5.50.

4. The softgel capsule of claim 1, wherein the pH dependent polymer is an acrylic polymer or a cellulosic polymer.

5. (canceled)

6. The softgel capsule of claim 4, wherein the acrylic polymer is an amino methacrylate copolymer.

7. softgel capsule of claim 1, wherein the shell composition comprises the pH dependent polymer in an amount (w/w) from about 1% to about 60%

8. The softgel capsule of claim 1, wherein the shell composition comprises the gelatin in an amount (w/w) from about 5% to about 75%

9. The softgel capsule of claim 1, wherein the shell composition further comprises a solubilizing agent.

10. (canceled)

11. The softgel capsule of claim 9, wherein the solubilizing agent comprises oxalic acid, malonic acid, succinic acid, glutaric acid, adipic acid, fumaric acid, maleic acid, phthalic acid, isophthalic acid, terephthalic acid, formic acid, acetic acid, propionic acid, butyric acid, valeric acid, cinnamic acid, lactic acid, benzoic acid, salicylic acid, gallic acid or toluic acid,

12. (canceled)

13. The softgel capsule of claim 9, wherein the shell composition comprises the solubilizing agent in an amount (w/w) from about 0.0001% to about 1%

14. The softgel capsule of claim 1, wherein the shell composition further comprises a plasticizer.

15. The softgel capsule of claim 14, wherein the shell composition comprises the plasticizer in an amount (w/w) of about 5 wt % to about 50 wt %

16. The softgel capsule of claim 14, wherein the plasticizer comprises glycerol, glycerin, sorbitol or combinations thereof.

17. The softgel capsule of claim 1, wherein the gelatin comprises Type A gelatin, Type B gelatin or mixtures thereof, or
wherein the gelatin is selected from the group consisting of fish gelatin, hide gelatin, bone gelatin or mixtures thereof.

18. (canceled)

19. The softgel capsule of claim 1, wherein at least one of:
the liquid medium is water,
the polyol is glycerol,
the glycol is polyethylene glycol, or
the alcohol is ethanol.

20-22. (canceled)

23. The softgel capsule of claim 1, wherein at least one of:
the fill composition comprises the liquid medium in an amount of at least 20%, or
the fill composition comprises the liquid medium in an amount of from 5% to 50%.

24. (canceled)

25. The softgel capsule of claim 1, wherein at least one of:
the capsule disintegrates in less than about 60 minutes in a gastric environment based on a disintegration test performed in a USP Apparatus II with paddles at a speed of 50 rpm in pH 1.2 buffer, or
the capsule disintegrates in at least about one hour in a basic medium based on a disintegration test performed in a basket-rack assembly NT-40H model apparatus in a 1000 mL beaker at about 37° C.±2° C.

26. (canceled)

27. The softgel capsule of claim 1, further comprising a water soluble active agent.

28. The softgel capsule of claim 1, further comprising levothyroxine or a pharmaceutically acceptable salt thereof.

29. The softgel capsule of claim 1, wherein the fill material comprises a pH of greater than about 4.

30. (canceled)

31. (canceled)
